(19) United States
(12) Patent Application Publication
(10) Pub. No.: US 2014/0096777 A1
(43) Pub. Date: Apr. 10, 2014

(54) Sleeping Device to Prevent Snoring

(71) Applicant: Nicole Derner, Los Gatos, CA (US)

(72) Inventor: Nicole Derner, Los Gatos, CA (US)

(21) Appl. No.: 13/867,999

(22) Filed: Apr. 22, 2013

Related U.S. Application Data
(60) Provisional application No. 61/636,437, filed on Apr. 20, 2012.

(51) Int. Cl. A61F 5/56

(52) U.S. Cl. CPC A61F 5/56
USPC 128/845

(57) ABSTRACT

A device configured to reduce snoring that includes a platform configured to elevate the torso of a user and a pillow mounted to the wedge platform and configured to elevate the head of the user.

### RELATED APPLICATIONS

This application claims priority to U.S. Application No. 61/636,437, filed on Apr. 20, 2012, which is hereby incorporated herein by reference in its entirety.

### BACKGROUND

Snoring is a chronic problem with many people, causing discomfort and exhaustion for those who snore, and often include those who sleep next to them. Many devices exist to address this issue, including devices that insert in nasal cavities, adhesive products that glue to the outside of the nose, and other devices that attempt to modify breathing passages in the nose. These devices are often invasive, uncomfortable, and simply inconvenient to use. There are also surgical approaches, but those are also not entirely proven and are even more invasive.

What is desired and needed in the art is a solution that is comfortable, non-invasive and convenient product solution that prevents or reduces snoring. As will be seen, the invention provides such a product solution in an elegant manner.

### BRIEF DESCRIPTION OF THE DRAWINGS

FIG. 1 shows a diagrammatic view of one embodiment of a sleeping device to prevent snoring.

FIG. 2 illustrates a perspective view of the embodiment of FIG. 1.

FIG. 3 illustrates another embodiment of a sleeping device to prevent snoring where a type of pillowcase separately holds two structures in place.

FIG. 4 illustrates one embodiment of a sleeping device to prevent snoring where a one piece product is provided configured to elevate and support upper torso while sleeping.

FIG. 5 illustrates another embodiment of a sleeping device to prevent snoring where a one piece product is provided.

FIG. 6 illustrates yet another embodiment of a sleeping device to prevent snoring where a one piece product is provided.

### DETAILED DESCRIPTION

The invention is directed to a device to reduce or eliminate snoring that is configured to elevate the upper torso at least about the level of the waist area and to support a person's upper torso and provide a head rest while sleeping. The headrest may be configured to support only the head, or alternatively may support the head and neck to comfortably position the head and neck with respect to the elevated torso in a manner that prevents snoring.

One embodiment includes a wedge platform configured to elevate the torso of a user and a pillow mounted to the wedge platform and configured to elevate the head of the user.

In one embodiment, provided includes a wedge platform configured to elevate the torso of a user and a pillow mounted to the wedge platform and configured to elevate the head of the user. The wedge platform may be made with a firm cushion material, or a flexible, semi-flexible, semi-firm, or composite material that provides elevated comfort to a user to they can be held in a consistent and comfortable elevated position while sleeping.

Different embodiments are possible within the invention. For example, the wedge platform may be made with a soft cushion material, a rigid material, a firm structure, a memory cushion, firm cushion, or other material that allows a user to elevate the user's torso. Also, the pillow may be made with a soft cushion material, a rigid material, a firm structure, a memory cushion, firm cushion, or other material that allows a user to elevate the user's head. The ILD is the measure of firmness, and many different materials exist that may be used to construct one or more structures that make up the novel device. Density is the measure of a specific ASTM test taken at various depths using an international rating system. Density of a material used in a particular structure determines the amount of chemical used to achieve a specific feel or function for a specific application, and it affects cost. Density may also vary throughout a structure, and can affect the manner in which a device is designed, manufactured, and how it may perform during use. The higher the density the more the chemical—the more the chemical the higher the cost. It is a linear calculation. In one example, cut foam is defined by the method in which the foam is fabricated. The foam is cut or convoluted to meeting specific designs. In one example of cut foam configuration a visco-elastic foam using a 2.5 density with an ILD of 12 may be used.

If a bi-component pillow using a base for support (memory foam has no support), one example of foam is Omalon® 2.15 density with a 20 ILD. Those skilled in the art may employ or design structures with a number of densities to achieve various feels and to accommodate the primary function.

Molded foam is one type of material used in such structures and is defined by the way chemical is converted into shapes and designs. The molded foam may be configured to facilitate any most shape. Molded foams by nature have a higher level of support even using memory foam. Because of the nature of molding a thin skin forms on the surface that gives it an enhanced ILD. We punch holes in these foams for pillows to help open the cells and to increase airflow. This also makes the pillow a bit softer.

In one example of a molded foam, a 2.7 density visco-elastic foam with an ILD of 7 may be used to configure a structure for this application. Alternatively, a 5.0 density visco-elastic foam with an ILD of 6 could be employed. Those skilled in the art will understand that, given this disclosure, there are many combinations and permutations of existing structures that may be combined in a similar or same manner as the embodiments described herein.

Still further, the pillow may be removeably attached to the wedge platform. Alternatively, the pillow may be removeably attached to the wedge platform with a Velcro fastener. Or, the pillow may be removeably attached to the wedge platform with a pillow case configured to hold the wedge platform and the pillow. The pillow may alternatively be fixedly attached to the wedge platform with a fastener, such as a button, Velcro, or other fastener.

In yet another embodiment, the pillow may be fixedly attached to the wedge platform with a common mold material, where the pillow is commonly molded with the wedge platform.

The pillow may include an overlapping ridge configured to accommodate a user's shoulder when lying on the wedge in a sideways position. Alternatively, the pillow may be configured in a trapezoidal shape that includes an overlapping ridge configured to accommodate a user's shoulder under the ridge when lying on the wedge in a sideways position with the user's head on the pillow.

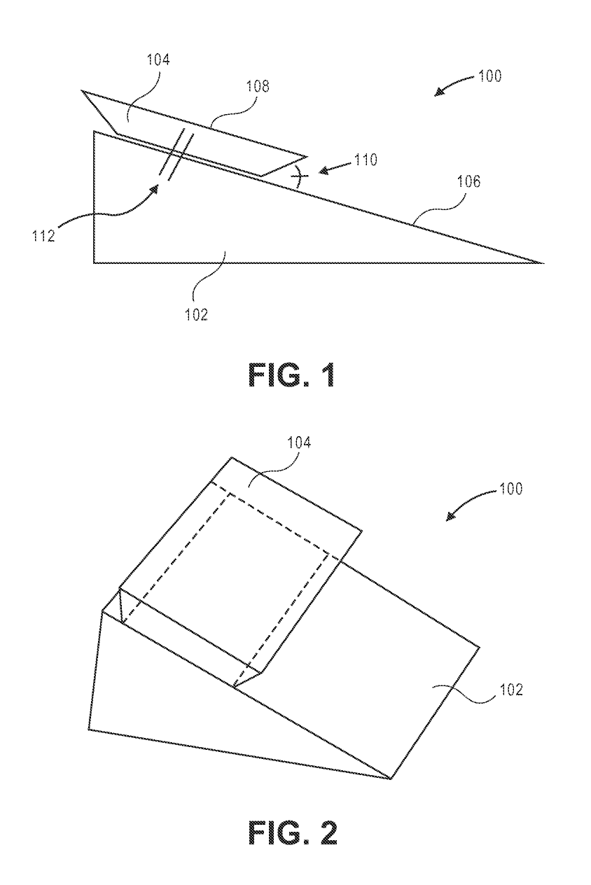

Referring to the figures, FIG. 1 shows a diagrammatic view of one example of a snore solution, where device 100 includes an elevation platform, shown in this example as a wedge 102. The wedge shown here is but one example of a platform, and it is contemplated that other shapes may be used in order to elevate a user's torso in a manner to help reduce snoring. This example also includes a head elevation platform, shown in this example as cushion 104 mounted atop surface 106. A user may rest its head upon surface 108, giving elevation and comfort while sleeping. There may be an inward angled portion 110 configured to accommodate a user's shoulder while sleeping on a side.

The cushion 104 may optionally be fastened to platform 102 via some type of fastening means 112, which may include Velcro™ fasteners, button/hole, or other fastening device. Furthermore, the two structures 102,104 may be manufactured as a single mold, where the different cushions are formed as a unitary device.

FIG. 2 illustrates a perspective view of the example 100 of FIG. 1, where the wedge structure 102 has the cushion 104 mounted thereon. It will be apparent to those skilled in the art that the cushion 104 may be formed in different shapes or may incorporate well known therapeutic or comfort oriented pillows.

For example, Posturepedic™ pillows are designed for comfort on a flat lying mattress, where the position of a person's head may be set over the mattress in a manner that is comfortable and proper for the user's health and comfort.

In one example, a posture-friendly pillow is configured on the wedge structure 102 in a manner that allows a user to place the user's head on the pillow while also having the user's torso elevated by the wedge structure, elevating the user's head with respect to the wedge structure.

In another example, a pillow that allows a user to have the user's torso elevated, while elevating the head, and also allowing the user to tuck the user's shoulder under the edge of the pillow gives the user a comfortable alternative, where the user may place the user's side of the face and head on the pillow while sleeping on the user's side. This allows a user to sleep sideways while being elevated at the torso and separately elevating the head.

Different shapes of the pillow may be provided in different examples, including an upside down trapezoidal shaped cross section, where a user may tuck the user's shoulder under an edge of the pillow while resting the user's head on the pillow. In other examples, a round, oval, cubic, ellipsoid, polygonal, vertex, triangle, parallelogram, rhombus, pentagon, hexagon, heptagon, octagon, nonagon, decagon, circle, or custom shaped therapeutic pillow may reside on the wedge platform to give the user comfort by comfortably resting the user's head while having an elevated torso. The pillow may be concave or convex, and may take on different shapes with different comfort effects. Any shaped pillow may be mounted or otherwise placed on the wedge platform, including formable pillows that fit to the head when it rests on the pillow and again when it moves while the user sleeps. The invention is not limited to any particular shape, and includes any possible shape of a pillow that is mounted or otherwise placed on a wedge shaped platform to give a user a comfortable to rest the users' head while the torso is elevated.

Furthermore, the wedge shaped platform may also take on other forms that provide elevation of the user's torso for comfort. Platforms may include a formable pillow that forms to the user's torso while elevating it, and may be relatively rigid, soft, or adaptable to the user's shape in response to the user resting on the platform. Thus, most any shape may be employed as the torso's platform, where the user's torso is elevated, while the user's head rests on the pillow or like structure located on the platform.

Still further, the pillow and platform may be formed in one single structure, where the shape of the unitary structure is configured to elevate the user's torso and provide a resting place for the user's head.

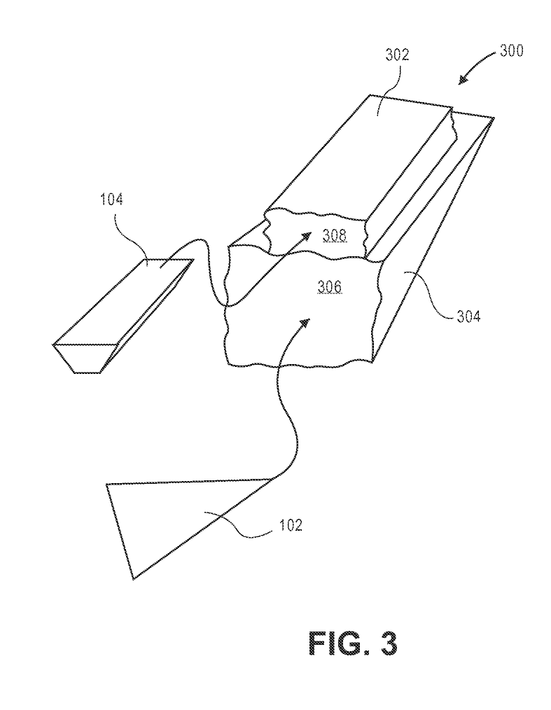

Still further, the two structures 102, 104 may be held in place with a type of pillowcase that separately holds the two structures in place, an example of which is illustrated in FIG. 3. In this embodiment, the wedge structure 102 and pillow 104 maybe be inserted into structure 300 having a wedge holder 302 and wedge holder 304, and also has openings 306 and 308 to receive the respective structures. Such a pillow case may hold different types of pillow shapes and sizes, and may even be configured with stretchable fabric to accommodate different shapes while securely holding the shapes together so that a user will be able to comfortably rest on the resulting structure and be properly configured for comfortable rest.

In another embodiment, the range of angle that the wedge may take approximately 2-35 degrees, or an elevation of 4 inches to 30 inches at the shoulder of the user. These factors may vary depending on age, height, weight, and other characteristics. Different elevations may vary based on condition as well, whether the user is recovering from surgery, reducing snoring, reducing acid reflux, or seeking other types of comfort. Certain angles may be useful for physical comfort, such as post-surgical or physical therapy based positions. Other angles may be useful for relief from snoring, sleep apnea, acid reflux, or other condition that may be cured by placing a person in the proper elevated and angled position while sleeping. Also, the attached or monolithic structure may elevate the torso at one angle, and the head at another angle or the same angle. The length of the wedge or the pillow may be different for different users, where a larger or taller person may have different sizes than smaller or shorter people. And, each structure or monolithic structure may position a person in different ways, where different conditions or desired results may require different configurations of the structures.

Also, the proper placement of a person's head may be useful to reduce snoring, apnea, acid reflux or other condition, wherein the secondary cushion such as an attached pillow on the wedge or similar shaped elevated structure that allows a person to properly place their head while being elevated on an angle. For example, a person's head may be positioned in a manner that elongates the user's neck, rather than allowing the head to hang back or forward while sleeping with the torso angled on the elevated structure. This may allow a user to properly open up their breathing passages so that snoring, apnea, acid reflux or other condition is lessened or eliminated. Those skilled in the art will understand that, given this disclosure, many different structures may be configured to accomplish such relief mechanisms that allow a user to be placed in a position that relieves these conditions.

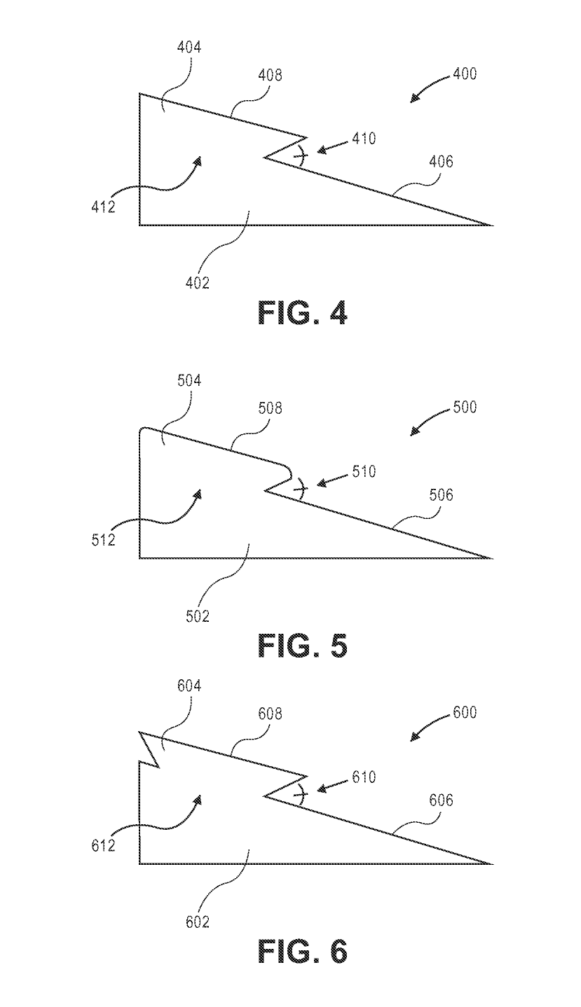

In another embodiment, a one piece product is provided with unique shape configured to elevate the upper torso at least at the level of the waist area and to support upper torso while sleeping. The angle at which it is elevated with respect to a horizontal base may vary. This is illustrated in FIGS. 4-6. This embodiment is similar to the embodiment discussed above, but it further is constructed in a unitary device. The one piece product also comfortably supports the head. The product may support only the head, or alternatively may support the head and neck to comfortably position the head and neck with respect to the torso in a manner that prevents snoring. In one example, a uniquely designed head rest has trapezoidal or curved edges that allow for the shoulder to wedge into the gap, thereby elongating the neck, which provides a means to reduce snoring.

In another embodiment, special and unique foam formulas that are used in the construction of the unique devices may further aid in reducing snoring. The elevated component may be a softer and more comfortable material to allow for ease of sleep while the head rest is relatively more firm for support and maintaining the head and neck in place. Cut foam or molded foam may be used in any of the above embodiments, and those skilled in the art will understand that together with this disclosure, many different variations, embodiments and examples are possible within the scope of the invention.

The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

1. A device, comprising:
a wedge platform configured to elevate the torso of a user; and
a pillow mounted to the wedge platform and configured to elevate the head of the user.

2. A device according to claim 1, wherein the wedge platform is made with a firm cushion material.

3. A device according to claim 1, wherein the wedge platform is made with a soft cushion material.

4. A device according to claim 1, wherein the wedge platform is made with a rigid material.

5. A device according to claim 1, wherein the wedge platform is made with a firm structure.

6. A device according to claim 1, wherein the wedge platform is made with memory cushion material.

7. A device according to claim 1, wherein the pillow is made with a firm cushion material.

8. A device according to claim 1, wherein the pillow is made with a soft cushion material.

9. A device according to claim 1, wherein the pillow is made with a rigid material.

10. A device according to claim 1, wherein the pillow is made with a firm structure.

11. A device according to claim 1, wherein the pillow is made with memory cushion material.

12. A device according to claim 1, wherein the pillow is removeably attached to the wedge platform.

13. A device according to claim 1, wherein the pillow is removeably attached to the wedge platform with a Velcro fastener.

14. A device according to claim 1, wherein the pillow is removeably attached to the wedge platform with a pillow case configured to hold the wedge platform and the pillow.

15. A device according to claim 1, wherein the pillow is fixedly attached to the wedge platform.

16. A device according to claim 1, wherein the pillow is fixedly attached to the wedge platform with a fastener.

18. A device according to claim 1, wherein the pillow is fixedly attached to the wedge platform with a button fastener.

19. A device according to claim 1, wherein the pillow is fixedly attached to the wedge platform with a common mold material.

20. A device according to claim 1, wherein the pillow is commonly molded with the wedge platform.

21. A device according to claim 1, wherein the pillow includes an overlapping ridge configured to accommodate a user's shoulder when lying on the wedge in a sideways position.

22. A device according to claim 1, wherein the pillow is configured in a trapezoidal shape that includes an overlapping ridge configured to accommodate a user's shoulder under the ridge when lying on the wedge in a sideways position with the user's head on the pillow.

23. A monolithic molded device, comprising:
a wedge platform configured to elevate the torso of a user; and
a pillow integrated with the wedge platform and configured to support the head of the user.
